(19) United States
(12) Patent Application Publication
(10) Pub. No.: US 2021/0181144 A1
(43) Pub. Date: Jun. 17, 2021

(54) GAS MULTISENSOR AND DEVICE FOR ANALYZING A MULTI-COMPONENT GAS MIXTURE

(71) Applicant: PRINTED ELECTRONICS TECHNOLOGIES LIMITED LIABILITY COMPANY ("PRINTELTECH" LLC), Moscow (RU)

(72) Inventors: Alexey Sergeevich SIZOV, Novocherkassk (RU); Daniil Sergeevich ANISIMOV, Moscow (RU); Askold Albertovich TRUL, Zhukovsky (RU); Victoriya Petrovna CHEKUSOVA, Otradny (RU); Aleksandr Anatolevich PERMYAKOV, Moscow (RU); Aleksey Nikolaevich KISILEV, Ivanovo (RU); Aleksey Andreevich VASILEV, Moscow (RU); Elena Valerievna AGINA, Moscow (RU); Sergey Anatolevich PONOMARENKO, Moscow (RU)

(73) Assignee: PRINTED ELECTRONICS TECHNOLOGIES LIMITED LIABILITY COMPANY ("PRINTELTECH" LLC), Moscow (RU)

(21) Appl. No.: 16/976,483

(22) PCT Filed: Mar. 1, 2018
(86) PCT No.: PCT/RU2018/000123

§ 371 Date: Mar. 4, 2021

(30) Foreign Application Priority Data
Feb. 28, 2018 (RU) 2018107433

(51) Int. Cl. G01N 27/414; H01L 27/28; H01L 51/05; H01L 51/00; G01N 33/00

(52) U.S. Cl. CPC G01N 27/4141; H01L 27/283; H01L 51/0545; G01N 33/0031; H01L 51/0084; H01L 51/0092; H01L 51/0083; H01L 51/0566

(57) ABSTRACT

The invention relates to the field of measuring equipment, and more particularly, to gas analysis sensors/chemical sensors designed to analyze the composition of gas mixtures and to detect and quantify toxic chemical gaseous compounds in an environment. The gas multisensor includes an array of N organic field-effect transistors, each of which consist of at least a source electrode and a drain electrode separated by an organic semiconductor layer, a gate electrode, a dielectric layer, and an additional receptor layer based on a metalloporphyrin of general formula 1 or 2 and completely or partially covering the organic semiconductor layer, while a metal ion M of metalloporphyrin is a transition metal, and each of the N organic field-effect transistors contained in the array differs from the other organic field- effect transistors in the array by the chemical structure of the receptor layer. The technical result is lowering the limit of detection of an electronic nose device based on chemosensors.

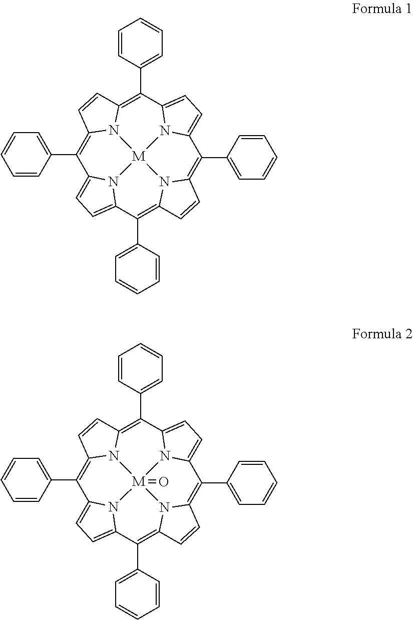

### FIELD OF THE INVENTION

The invention relates to the field of measuring technology, in particular, to gas sensors—chemical sensors designed for the analysis of gas mixtures, the detection and quantitative determination of toxic chemical gaseous compounds in the environment.

More specifically, it relates to “Electronic nose” systems, representing itself as an artificial olfactory system based on an array of chemical sensors, each of which is an organic field-effect transistor with a limited specificity.

The claimed device for a multicomponent gas mixture analyzing—“Electronic nose”—based on organic field-effect transistors is intended for a wide range of practical applications and to replace the human olfactory system by creating intelligent automated systems for air control and odor detection. In particular, “Electronic Nose” can be used to determine the quality of certain categories of high-value food products, such as coffee, tea, olive oil or wine in production and retail chains [Taurino A. M., Zuppa M., Presicce D. S. [et. al.] Miniaturized Hybrid System for Olive Oil Evaluation/2005 Ieee Sensors, Vols 1 and 2.-2005. -P. 1022-1025]. Another application of such systems is non-invasive medical diagnostics of certain types of diseases, such as diabetes [Ping W., Yi T., Xie H. B., Shen F. R. A Novel Method for Diabetes Diagnosis Based on Electronic Nose/Biosensors & Bioelectronics.-1997.—V. 12, No 9-10. -P. 1031-1036] or asthma [Bates C. A., Silkoff P. E. Exhaled Nitric Oxide in Asthma: From Bench to Bedside/Journal of Allergy and Clinical Immunology.-2003.-V. 111, No 2.-P. 256-262]. A promising application of the “Electronic Nose” is also monitoring the quality of ambient air in living and working areas [McNabola A., Broderick B. M., Gill L. W. A Principal Components Analysis of the Factors Effecting Personal Exposure to Air Pollution in Urban Commuters in Dublin, Ireland/Journal of Environmental Science and Health Part a-Toxic/Hazardous Substances & Environmental Engineering.-2009.-V. 44, No 12.-P. 1219-1226], as well as fire control [Scorsone E., Pisanelli A. M., Persaud K. C. Development of an Electronic Nose for Fire Detection/Sensors and Actuators B-Chemical.-2006.-V. 116, No 1-2.-P. 55-61].

### BACKGROUND

The organic field-effect transistors usage for the “Electronic nose” system is the most promising solution, since it allows combining high sensitivity, low power consumption, as well as the possibility of each single sensor included in “Electronic nose” modification by a receptor layers of various chemical structures for increasing the selectivity of the sensors used. An additional advantage of the multisensor, which is the basis of the “Electronic Nose”, is its low cost allowing disposable usage of the device what is relevant in the case of biomedical applications.

It is known an electronic nose or electronic tongue, including a single substrate with multiple sensitive areas. Each of the receptor areas contains a mixture of several different receptors in varying proportions. Area's physical parameters changes during interactions with a sample, what allows to determine the presence of a detectable compound by optical, fluorescence, or confocal microscopy as well as by surface plasmon resonance or by measuring impedance [application EA201491561 A1, publication date 2015 Jan. 30].

The disadvantage of this sensor is the usage of complex methods for signal registration, which exclude the portable devices creation possibility, as well as the fabrication of a receptors mixture in various proportions leads to over cost of the final device.

Also it is known an electronic nose based on an array of field-effect transistors, in which unoxidized nanotubes covered with various functional groups other than methyl are used as a semiconductor layer.

The known device is designed to detect volatile organic compounds at concentrations below 1 ppm. The electrical signal received from each individual sensor in the electronic nose is analyzed using a measuring unit (pattern recognizer) in accordance with one of the known data analysis methods, such as the principal component analysis or an artificial neural network algorithms. The device can be used for non-invasive diagnostics of medical diseases, including cancer [US application 2010/0198521 A1, publication date May 8, 2010].

The disadvantage of this electronic nose is the technical complexity of semiconductor layer fabrication, which requires precise single nanotube placement to the electrodes, as well as is the possibility of limited amount of detectable chemical compounds (volatile organic compounds).

It is known a multi-channel “electronic nose” based on piezosensors coated with various sorbents. The device allows to analyze samples with various compositions, including very similar to each other, containing microconcentrations of determined [patent RU2327984 C 1, published Feb. 19, 2007].

The disadvantage of the described device is large electrical system for the individual gas sensors response measuring, which excludes the portable gas analysis systems creation possibility.

It is known a gas mixture composition analyzing device, which includes a dielectric substrate with heating elements covered by gas-sensitive metal-oxide layer, strip-like electrodes located at the edges of the gas-sensitive metal-oxide layer and at least one measuring electrode located between strip electrodes. The strip electrodes the measuring have the leads for connection to a voltage source and potential measuring device, correspondingly. The device allows to distinguish individual volatile organic compounds [patent RU2392614 C1, publication date Mar. 6, 2009].

The disadvantage of the device is the necessity of to the metal-oxide layer heating to high temperatures (100-400° C.), what excludes the possibility of creating energy-efficient systems on its basis. In addition, the device has limited selectivity.

It is known a method for sensor matrix creation based on piezoelectric quartz resonators, the electrodes of which are modified by various chemical sorbents. The gas mixture fingerprint is formed from the individual sensors responses and then analyzed [patent RU2442158 C2, publication date Apr. 23, 2010]. The disadvantage of devices is the limited set of detectable compounds due to the limited variety of sorbents used.

It is known field-effect transistor with a protein containing lipid membrane, covering the drain, source as well as a semiconducting channel. The semiconducting channel is a nanostructured semiconductor based on nanotubes, nanowires, or is a nanometer-thick film. The device can be used in the “Electronic nose” systems and is capable of detecting biological origin compounds with high sensitivity [U.S. Pat. No. 8,377,706 B2, published Feb. 19, 2013].

The disadvantage of the described device is the limited range of detectable compounds, including only biological origin compounds, but not including low molecular weight toxic gases.

It is known field-effect transistor based on silicon, which has the upper and lower gates, as well as the receptor layer. An array of sensors based on such field-effect transistors with different receptor layers can be combined into an “Electronic nose” system [application US2011/0108892 A1, publication date May 12, 2011].

The disadvantage of the described device is the presence of two gate electrodes—the upper and the lower complicating the sensor and the electronic nose based on its creation.

It is known an “electronic nose” based on sensors of one from several types (including transistors), while the semiconductor layer consists of columnar discotic liquid crystals nanofilaments. In addition, individual sensors are insensitive to water vapor. The device is designed to detect volatile organic compounds with high sensitivity [application US 2010/0191474 A1, publication date Jul. 29, 2010].

The disadvantage of the described device is the limited range of detectable compounds, including only volatile organic compounds, but not including low molecular weight toxic gases.

The closest to the claimed technical solution is an “electronic nose”, in which chemisorption sensors with different metalloporphyrins sensitive layers, differing from each other by the central metal atom, are used as separate sensors. The principle of device operation is based on measuring the optical characteristics of individual sensors, subsequent analysis of the data obtained using known processing methods, such as principal component analysis, and comparing the obtained characteristics with known calibration curves. The device is capable of detecting low molecular weight toxic gases such as ammonia, nitrogen oxides, carbon monoxide and triethylamine [Filippini D., Alimelli A., Di Natale C., Paolesse R., D'Amico A., Lundstrom I. Chemical Sensing with Familiar Devices/Angew Chem Int Ed Engl.-2006.-V. 45, No 23.-P. 3800-3].

The disadvantage of this device is its insufficient sensitivity (above 2 ppm in the case of ammonia, above 20 ppm in the case of nitrogen), due to the weakly sensitive method of individual sensors response measuring.

### SUMMARY OF THE INVENTION

The technical problem to be solved by the claimed invention consists in creating a highly sensitive “electronic nose” device for multicomponent gas mixture analyzing, as well as for determining the concentrations of various low molecular weight toxic gases in the gas mixture composition in a concentrations less than 1 ppm.

The technical result achieved by the claimed invention implementation is decreasing of the limit of detection of an “electronic nose” devices based on chemisorption sensors with receptor layers based on various metalloporphyrins, differing from each other by the central metal atom.

The technical result is achieved by usage of gas multisensory, which includes an array of N organic field-effect transistors consisting of at least an electrodes—“drain”, “source” separated by an organic semiconductor layer, a “gate” electrode, a dielectric layer and an additional receptor layer based on metalloporphyrin of general formula 1 or 2, completely or partially covering the organic semiconductor layer of the N-th organic field-effect transistor:

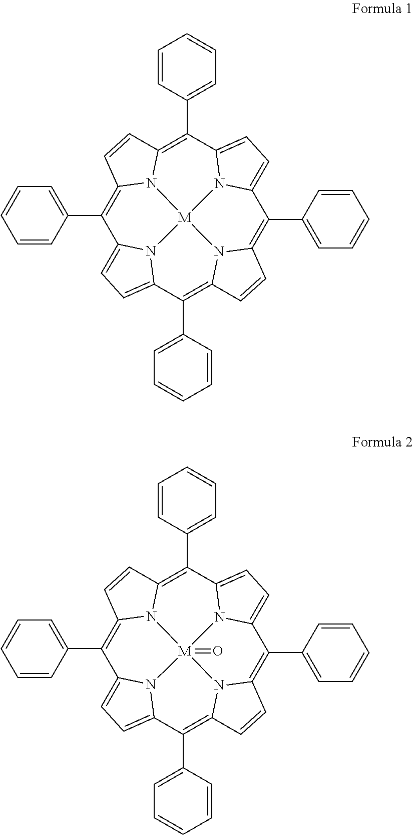

where the metal-ion of porphyrin M is a transition metal, and the each of the N organic field-effect transistors differs from other organic field-effect transistors in the array by the chemical structure of the receptor layer.

In addition, in the particular case of the invention implementation, the metal ion of porphyrin M is a transition metal ion Cu, Zn, Co, Fe, Ni, Cr, Mn, Ti or V.

In addition, in the particular case of the invention implementation, an array of N organic field-effect transistors is formed on a single substrate.

In addition, in the particular case of the invention implementation, the gas multisensor is designed to determine the presence of one of the following gases: ammonia, hydrogen sulfide, nitrogen dioxide, ethyl mercaptan.

Also, The technical result is achieved by usage of gas multisensory, which includes an array of N organic field-effect transistors consisting of at least an electrodes—“drain”, “source” separated by an organic semiconductor layer, a “gate” electrode, a dielectric layer and an additional receptor layer based on metalloporphyrin of general formula 1 or 2, completely or partially covering the organic semiconductor layer of the N-th organic field-effect transistor:

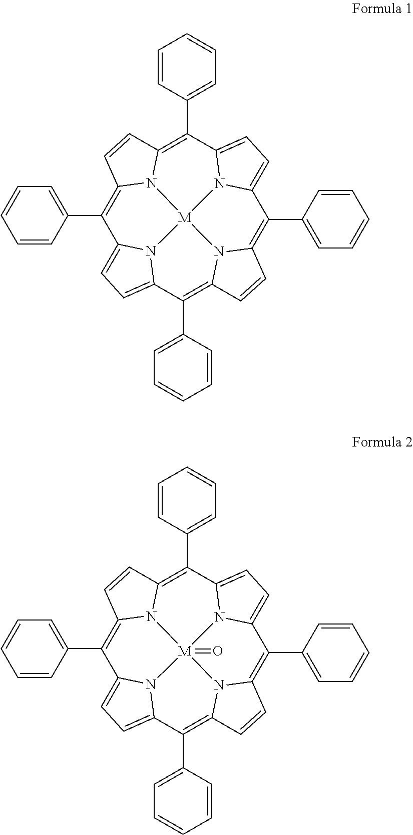

where the metal-ion of porphyrin M is a transition metal, and each of the N organic field-effect transistors differs from other organic field-effect transistors in the array in the chemical structure of the receptor layer, while the array of N organic field-effect transistors contains at least one organic field-effect transistor without a receptor layer; designed for quantitative determination of the concentration of several low molecular weight toxic gases in the atmospheric air or gas mixture.

In addition, in the particular case of the invention implementation, the array of N organic field-effect transistors on a single substrate creation is done by placing an organic field-effect transistors with receptor layers mainly along the perimeter of the substrate, while an organic field-effect transistors without a receptor layer are placed mainly in the central part of the substrate.

In addition, in the particular case of the invention implementation, the receptor layers of metalloporphyrins are deposited using the Langmuir-Blodgett method by partially dipping the substrate into a subphase.

In addition, in a particular case of the invention implementation, the organic semiconductor layer of the organic field-effect transistor is a self-organizing monolayer fabricated from chemically inert organosilicon oligothiophene, benzothienobenzothiophene or diphenylbitiophene derivatives soluble in organic solvents.

In addition, in the particular case of the invention implementation, the thickness of the organic semiconductor layer of the organic field-effect transistor is 2-20 nm. In addition, in the particular case of the invention implementation, a gas multisensor is desighed to determine the presence and concentration of one of the following gases: ammonia, hydrogen sulfide, nitrogen dioxide, ethyl mercaptan.

Also, the technical result is achieved by the “Electronic nose” device for analyzing a multicomponent gas mixture includes a gas multisensor, made according to any of the embodiments given above; a measuring unit connected to each of the organic field-effect transistors in the aforementioned gas multisensor, the measuring unit being configured to measure the current values of each N organic field-effect transistors included in the array, depending on time; a microprocessor connected to the measuring unit and configured to analyze the response of each N organic field-effect transistors and determine the type of low molecular weight toxic gas presented in the gas mixture, as well as its concentration; a tight chamber with gas inlet and outlet,where the aforementioned multisensor gas is taken place.

In addition, in the particular case of the invention implementation, the device is designed to determine the presence and concentration of one of the following gases: ammonia, hydrogen sulfide, nitrogen dioxide, ethyl mercaptan.

A decrease the limit of detection value of an “electronic nose” device based on chemisorption sensors with receptor layers based on metalloporphyrins of various chemical structures is achieved due to two factors. First, the chemisorption sensors used in the device are organic field-effect transistors, in which receptor layers based on metalloporphyrins of various chemical structures are deposited on top of the organic semiconductor layer, which allows to record the sensor response in the form of dependences of the current value on the applied voltage. Secondly, the films of the organic semiconductor and the metalloporphyrin-based receptor have the minimum possible thickness and, in a preferred embodiment, are monolayers of the substance, which corresponds to film thicknesses in the range from 2 to 20 nm.

### BRIEF DESCRIPTION OF THE DRAWINGS

An example of implementation of the invention is confirmed by the following drawings.

FIG. 1 is a diagram of a gas multisensor device based on organic field-effect transistors.

FIG. 2 is a diagram of a gas multisensor based on organic field-effect transistors placed on different substrates.

FIG. 3 is a diagram of a gas multisensor based on organic field-effect transistors manufactured on a single substrate.

FIG. 4 is a graph illustrating volt-ampere curves of an organic field-effect transistor before metalloporphyrin deposition.

FIG. 5 is a graph illustrating volt-ampere curves of an organic field-effect transistor after deposition of metalloporphyrin.

FIG. 6 is a diagram of an “electronic nose” device based on a gas multisensor.

FIG. 7 is a graph illustrating the dependence of the current value on time for an organic field-effect transistor with a receptor layer of TiO-TPP metalloporphyrin at different concentrations of one gas (ammonia).

FIG. 8 is a graph illustrating the the dependence of the current value on time for an organic field-effect transistor with a receptor layer of TiO-TPP metalloporphyrin for different gases of the same concentration.

FIG. 9 is a graph illustrating the dependence of the current value on time for an organic field-effect transistor with various receptor layers of metalloporphyrins.

FIG. 10 illustrates the results of recording the response of a gas multisensor based on organic field-effect transistors by the linear discriminant analysis (LDA) method.

FIG. 11 illustrates dependence of the relative value of the current in the organic field-effect transistor without the receptor layer on the gas concentration for a gas mixture containing ammonia (NH₃).

FIG. 12 illustrates dependence of the relative value of the current in the organic field-effect transistor without the receptor layer on the gas concentration for a gas mixture containing hydrogen sulfide (H₂S).

FIG. 13 illustrates dependence of the relative value of the current in the organic field-effect transistor without the receptor layer on the gas concentration for a gas mixture containing nitrogen dioxide (NO₂).

FIG. 14 illustrates dependence of the relative value of the current in the organic field-effect transistor without the receptor layer on the gas concentration for a gas mixture containing ethyl mercaptan (Et-SH).

In the drawings, the positions have the following designations:

1—gas sensor based on organic field-effect transistor with a receptor layer;

1¹—substrate of each organic field-effect transistor;

1²—single substrate for placing an array of organic field-effect transistors;

1³—organic field-effect transistor without a receptor layer;

2—drain electrode;

3—source electrode;

4—organic semiconductor layer;

5—gate electrode;

6—dielectric layer;

7—receptor layer;

8—gas multisensor based on organic field-effect transistors;

9—sealed chamber;

10—power supply;

11—measuring unit;

12—microprocessor.

### DETAILED DESCRIPTION OF THE INVENTION

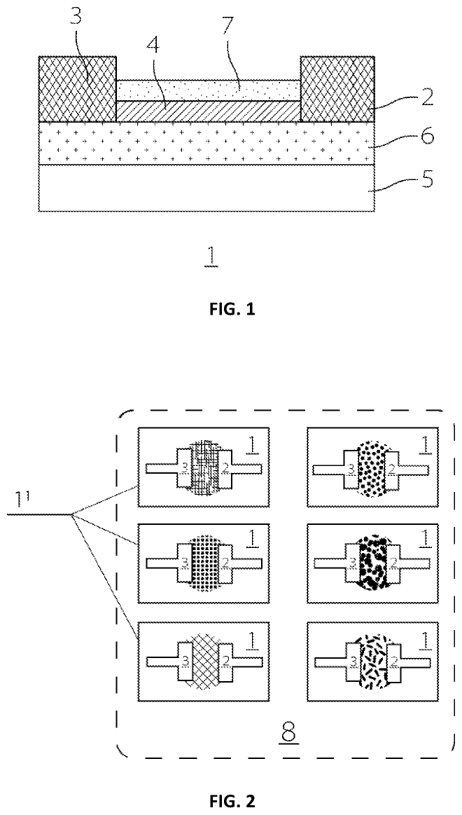

The gas multisensor 8 shown in FIG. 1 is an array of N organic field-effect transistors 1, each of which includes at least electrode 2—“drain”, electrode 3—“source”, separated by an organic semiconductor layer 4, electrode 5—“gate”, dielectric layer 6 and receptor layer 7, completely or partially covering the organic semiconductor layer 4 in the structure N of the organic field-effect transistor.

In a preferred embodiment of the invention, the receptor layer 7 is made on the basis of a metalloporphyrin of the general formula 1 or 2:

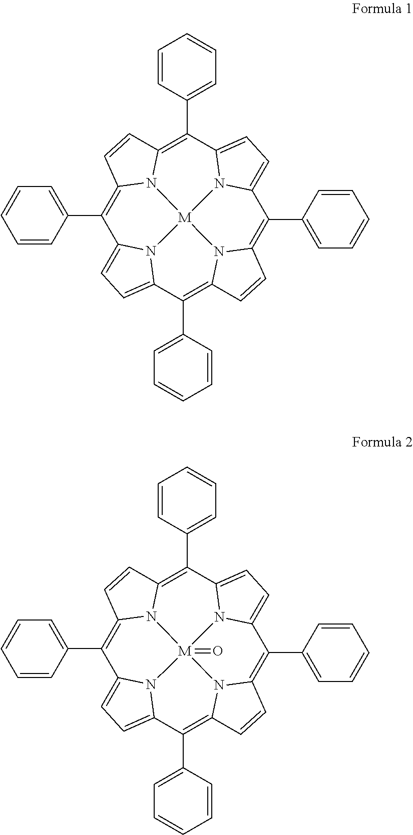

where the porphyrin metal ion M is the ion of transition metal, such as Cu, Zn, Co, Fe, Ni, Cr, Mn, Ti or V. In a preferred embodiment of the gas multisensor shown in FIG. 2, an array of N organic field-effect transistors 1, used for a qualitative analysis of the composition of atmospheric air or a gas mixture, may contain at least one organic field-effect transistor 1³ having no receptor layer, which can be used to quantify the concentration of low molecular weight toxic gas in atmospheric air or gas mixture, containing ammonia (NH₃), hydrogen sulfide (H₂S), nitrogen dioxide (NO₂), ethyl mercaptan (Et-SH).

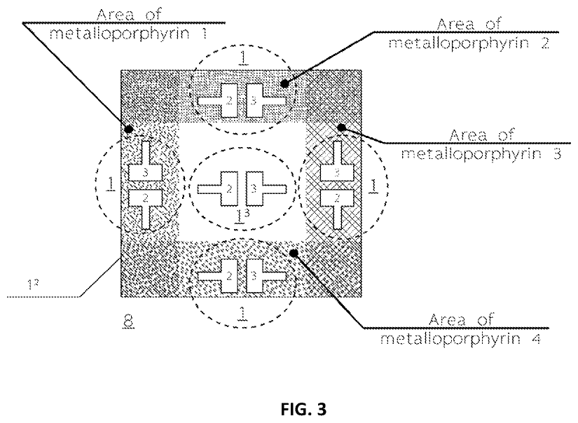

In a preferred embodiment of a gas multisensor, an array of N organic field-effect transistors 1 is formed on a single substrate 1² (FIG. 3), while the receptor layers 7 of metalloporphyrins are transferred using the Langmuir-Blodgett method by partially dipping the substrate 1² into a subphase. If there are N organic field-effect transistors 1 in the array of at least one organic field-effect transistor 1³ that does not have a receptor layer, it is preferable to place such a transistor in the central part of the substrate 1², and N organic field-effect transistors 1 with a receptor layer 7 are placed mainly along the perimeter of the substrate 1². The proposed arrangement of N organic field-effect transistors 1 allows the transfer of each subsequent receptor layer 7 without damaging the receptor layers 7 of other transistors in the array.

In a preferred embodiment of the gas multisensor 8, the organic semiconductor layer 4 is a self-assembled monolayer and can be made of chemically inert organosilicon derivatives of oligothiophenes, benzothienobenzothiophenes or diphenylthiophenes, such as 1,3-bis[11-(5′″-hexyl-2,2′:5′,2″:5″,2′″-quatrothiophen-5-yl)undecyl]-1,1,3,3-tetramethyldisiloxane, 1,3 -bis[11-([1]benzothieno [3,2-b][1]benzothien-2-yl)undecyl]-1,1,3,3-tetramethyldisiloxane, 1,3 bis[11-(7-hexyl[1]benzothieno[3,2-b][1]benzothien-2-yl)undecyl]-1,1,3,3 -tetramethyldisiloxane, 1,3-bis[11-(4-{5-[4(trimethylsilyl)phenyl]-2,2-bitien-5-yl}phenyl)undecyl]-1,1,3,3 -tetramethyldisiloxane. The implementation of the organic semiconductor layer 4 is not limited to the aforementioned examples.

Organic semiconductor layer 4 can be obtained by any of the known methods, in particular, by the Langmuir Blodgett method [Sizov A. S., Agina E. V., Gholamrezaie F. [et. al.] Oligothiophene-Based Monolayer Field-Effect Transistors Prepared by Langmuir-Blodgett Technique/Applied Physics Letters.-2013.-V. 103, No 4.-P. 043310], by the Langmuir-Schaefer method [Tanese M. C., Farinola G. M., Pignataro B. [et. al.] Poly(Alkoxyphenylene-Thienylene) Langmuir-Schäfer Thin Films for Advanced Performance Transistors/Chemistry of Materials.-2006.-V. 18, No 3.-P. 778-784], by spin coating method [Hall D. B., Underhill P., Torkelson J. M. Spin Coating of Thin and Ultrathin Polymer Films/ Polymer Engineering and Science.-1998.-V. 38, No 12.-P. 2039-2045], by doctor blade method [Yan Y., Huang L. B., Zhou Y. [et. al.] Self-Aligned, Full Solution Process Polymer Field-Effect Transistor on Flexible Substrates/Sci Rep.-2015.-V. 5.-P. 15770], by drop-casting method [Diao Y., Shaw L., Bao Z., Mannsfeld S. C. B. Morphology Control Strategies for Solution-Processed Organic Semiconductor Thin Films/Energy Environ. Sci.-2014.-V. 7, No 7.-P. 2145-2159].

In a preferred embodiment of the gas multisensor 8, the thickness of the organic semiconductor layer 4 can be from 2 to 20 nm, which provides high sensor sensitivity. Since the electric current in the N organic field-effect transistor, which is part of the gas multisensor 8, is localized in a thin near-surface layer at the boundary “organic semiconductor layer 4—dielectric layer 6”, the thickness of the organic semiconductor layer 4 in the range from 2 to 20 nm provides a direct effect of the receptor layer 7 on the current-carrying part of the semiconductor layer 4. The lower limit of the specified range of 2 nm corresponds to the minimum thickness of the organic semiconductor layer 4, at which the organic field-effect transistors 1 exhibit electrical and gas-sensitive properties. The upper limit of the specified range of 20 nm provides the ability to detect low concentrations of target gases in the concentration range of less than 1 ppm. With an increase of the thickness of the organic semiconductor layer 4, the sensitivity of the gas multisensor in the ppb range decreases.

In a preferred embodiment of the gas multisensor 8, the receptor layer 7 is a monolayer, which provides a high sensitivity of the device. When the detected toxic gas interacts with the surface of the receptor layer 7, local dipole moments arise, creating electrostatic fields. Since the magnitude of the field decreases with distance, the small thickness of the receptor layer 7 of metalloporphyrin provides the greatest influence of the receptor surface on the conducting channel of the transistor.

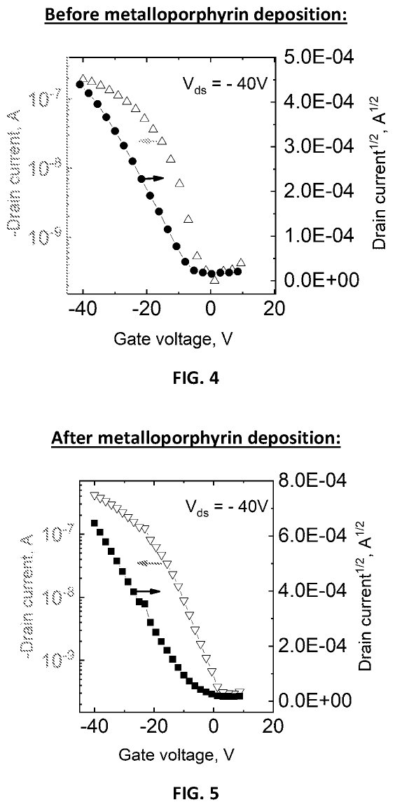

The necessity to use separate semiconductor 4 and receptor 7 layers stems from the fact that the receptor layer 7 with a small thickness has extremely low electrical characteristics, insufficient for the gas sensor operation. The principal factor for achieving the technical result is the preservation of high electrical characteristics of the monolayer organic field-effect transistor 1 during the transfer of the receptor layer 7. This result is achieved due to the fact that the receptor layer 7 is transferred to the substrate 1¹ or 1², containing N organic field-effect transistors 1, using the Langmuir-Blodgett method or Langmuir-Schaeffer method [Wei Z. M., Cao Y., Ma W. Z., Wang C. L., Xu W., Guo X. F., Hu W. P., Zhu D. B. Langmuir-Blodgett Monolayer Transistors of Copper Phthalocyanine/Applied Physics Letters.-2009.-V. 95, No 3], which excludes the ingress of the organic solvent into the organic semiconductor layer 4 and violation of its integrity. To illustrate the result, FIG. 4 shows the electrical characteristics of the organic field-effect transistor before applying the receptor layer 7, and FIG. 5 shows the electrical characteristics of the organic field-effect transistor after applying the receptor layer 7.

In a preferred embodiment of the gas multisensor 8, the dielectric layer 5 can be made of thermally grown dry silicon dioxide modified with a self-assembled monolayer (SAM-self-assembled monolayer) of octyldimethylchlorosilane (ODMS) or another alkylchlorosilane providing a sufficiently low surface roughness (<0.5 nm) of the dielectric layer 5.

The production of a gas multisensor 8 based on organic field-effect transistors 1 shown in FIG. 2 and FIG. 3 is preferably carried out as follows.

By known methods, for example, [Sizov A. S., Anisimov D. S., Agina E. V. [et. al.] Easily Processable Highly Ordered Langmuir-Blodgett Films of Quaterthiophene Disiloxane Dimer for Monolayer Organic Field-Effect Transistors/Langmuir.-2014.-V. 30, No 50.-P. 15327-34], on a single substrate 1² or on separate substrates 1¹ an array of N organic field-effect transistors 1 is formed, each of which includes at least electrode 2—“drain”, electrode 3—“source”, separated by layer 4 of organic semiconductor, electrode 5—“gate” and dielectric layer 6. Then, over the layer 4 of the organic semiconductor of each of the N organic field-effect transistors sequentially using the Langmuir-Blodgett or Langmuir-Schaeffer methods a receptor layers 7 of metalloporphyrins of various chemical structures are transferred.

As an example, the following compounds were used to form the receptor layer 7 based on metalloporphyrins:

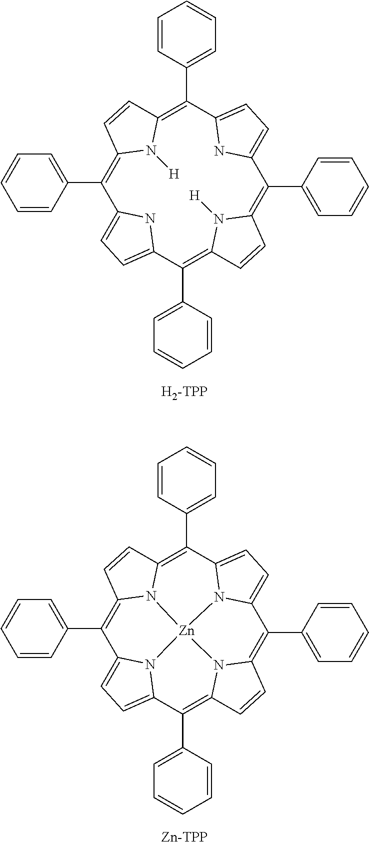

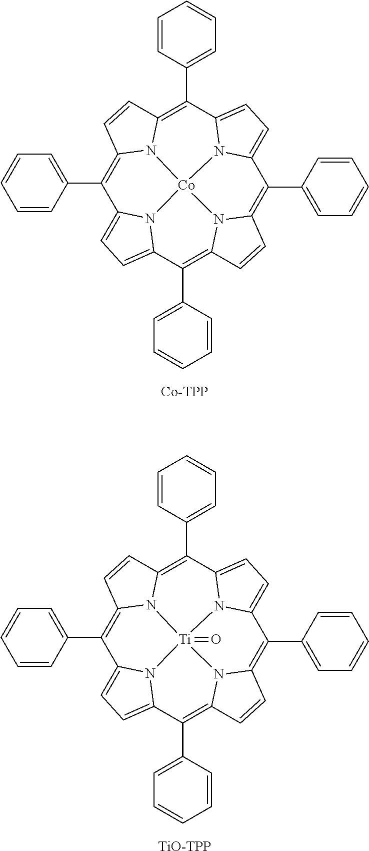

The technical result is achieved by using a number of 4-6 different metalloporphyrins having a general formula 1 or 2:

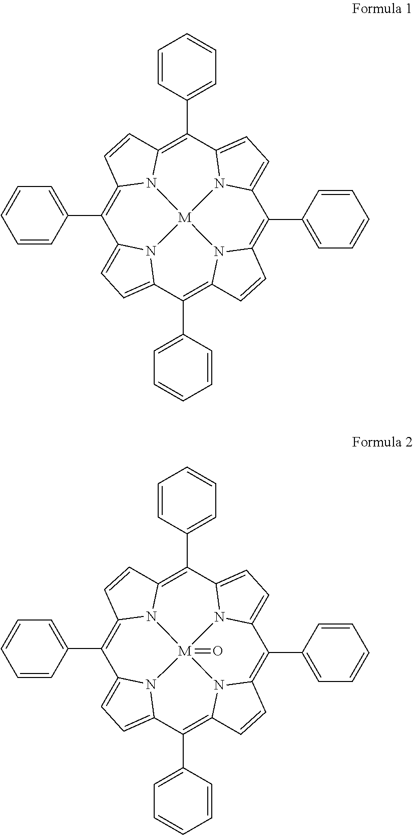

where the porphyrin metal ion M is the transition metal ions Cu, Zn, Co, Fe, Ni, Cr, Mn, Ti or V.

The choice of these metalloporphyrins is due to two factors. The first is the simplicity of their chemical formula, which does not contain side substituents, which simplifies their synthesis and reduces the cost of final materials and devices based on them. The second factor is that the porphyrins used are readily soluble in common organic solvents, such as toluene, and are capable of forming large area uniform monolayers on the water surface, which can be then transferred onto a solid substrate by the Langmuir-Blodgett method. The formation of a homogeneous large area receptor monolayer provides a high sensitivity of this layer to the presence of toxic gases, while any defects in the layer, especially its thickening, increase the sensor limit of detection.

At the same time, an increase in the amount of organic field-effect transistors 1, having different receptor layers 7, in the composition of the gas sensor array 8 increases the reliability of the qualitative analysis of the composition of atmospheric air or gas mixture.

In the case of the creation of an array of N organic field-effect transistors 1 on a single substrate 1² (FIG. 3), the receptor layers of metalloporphyrins are transferred using the Langmuir Blodgett method by partially dipping the substrate into a subphase.

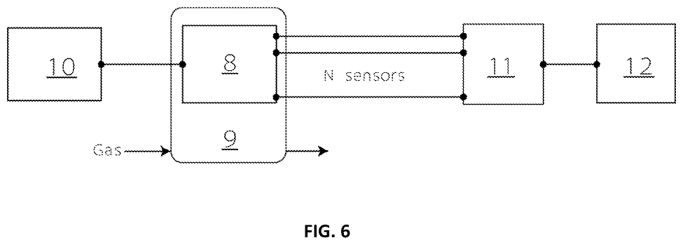

The “electronic nose” for analyzing a multicomponent gas mixture, shown in FIG. 6, includes a gas multisensor 8 based on organic field-effect transistors 1 and 1³ according to any of the embodiments described above, a microprocessor 9, a measuring unit 10 connected to electrodes 2 «drain», 3 «source» and 5 «gate» of each of the N organic field-effect transistors 1 and 1³ as part of the gas multisensor 8 and the microprocessor 12, a sealed chamber 9 with gas inlet and outlet, where the gas multisensor 8 is located, power supply 10, connected to electrodes 2 “drain”, 3 “source” and 5 “gate” of each of the N organic field-effect transistors 1 and 1³.

The operation of an “electronic nose” for analyzing a multicomponent gas mixture based on a gas multisensor 8, including an array of N organic field-effect transistors 1, is carried out as follows.

A gas mixture containing one of the detectable gaseous toxic compounds in concentrations from 10 ppb to 1 ppm is supplied to the sealed chamber 9. A constant negative potential V of the value selected so that the electric field in the dielectric layer 6 is at least 50 kV/mm and the electric field in the layer 4 of the organic semiconductor is at least 0.5 kV/mm, is supplied to the electrodes 2 “drain” and electrodes 5 “gate” of each of the N organic field-effect transistors 1 with receptor layers 7, which are part of the gas multisensor 8 (FIG. 2 or FIG. 3), using the power supply 10. Furthermore, the values of the current Iₖ (k=0 . . . N-1) are measured using the measuring unit 11 for each of the N organic field-effect transistors 1 with receptor layers 7 included in the multisensor 8, depending on the time t.

Furthermore, using software, the relative changes in current in N organic field-effect transistors are calculated in the microprocessor 12 according to the formula (1):

Iᵣₑₗ^{k}=I^{k}/I₀^{k}, (1)

where Iᵣₑₗ^{k}—relative current, relative units;
I^{k}—current in the channel of the N-th organic field-effect transistor at a given time, A;
I₀^{k}—initial current in the channel of the N-th organic field-effect transistor, A.

The resulting series of values Iᵣₑₗ^{k} (k=0 . . . N-1) is used as an input data for one of the known methods of data analysis, implemented using the microprocessor 12 according to a given algorithm. As such a method, well-known methods of data processing for “electronic nose” systems can be used: the method of principal components analysis (PCA), the method of linear discriminants analysis (LDA), a neural network, etc. [Jurs P. C., Bakken G. A., McClelland H. E. Computational Methods for the Analysis of Chemical Sensor Array Data from Volatile Analytes/Chemical Reviews.-2000.-V. 100, No 7.-P. 2649-2678; Pedregosa F., Varoquaux G., Gramfort A., Michel V., Thirion B., Grisel O., Blondel M., Prettenhofer P., Weiss R., Dubourg V., Vanderplas J., Passos A., Cournapeau D., Brucher M., Perrot M., Duchesnay E. Scikit-Learn: Machine Learning in Python/Journal of Machine Learning Research.-2011.-V. 12.-P. 2825-2830].

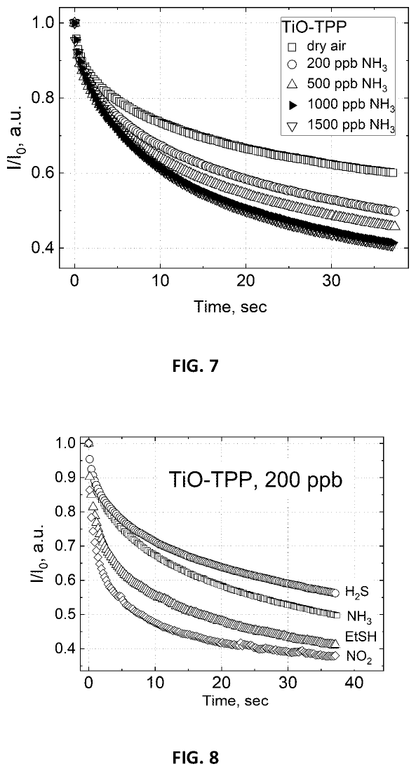

The possibility of using known methods for the analysis of the data obtained and for a qualitative analysis of the composition of atmospheric air or gas mixture is due to a combination of the following factors. First, the values Iᵣₑₗ^{k} for each of N organic field-effect transistors 1 with a receptor layer 7 of metalloporphyrin in the gas sensor 8 depend on the content and concentration of low-molecular-weight toxic gases in the atmospheric air or gas mixture. In this case, the values Iᵣₑₗ^{k} in dry air without ammonia and with its content of 200 ppb-1 ppm differ significantly (see FIG. 7).

Secondly, the values Iᵣₑₗ^{k} for each of the N organic field-effect transistors 1 with a receptor layer 7 of metalloporphyrin depend on the type of low molecular weight toxic gas present in the atmospheric air or gas mixture. This fact is due to various properties of toxic gas molecules, such as size, magnitude of the dipole moment, donor-acceptor properties, etc. For example, FIG. 8 shows the dependence of the current value on time for an organic field-effect transistor with a receptor layer 7 of metalloporphyrin TiO-TPP for different gases of the same concentration.

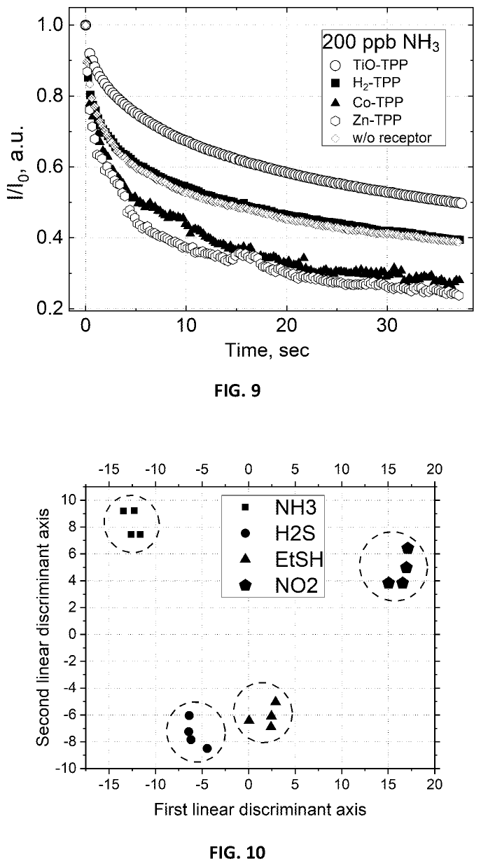

Third, the values Iᵣₑₗ ^{k} for each of N organic field-effect transistors 1 with a receptor layer 7 of metalloporphyrin, in the presence of a low molecular weight toxic gas, depend on the central atom of the metalloporphyrin molecule. This fact is due to differences in the kinetics of sorption of the same gas on the surface of a layer of metalloporphyrins of different chemical structure. For example, in FIG. 9 shows the dependence of the current value on time for an organic field-effect transistor with different receptor layers 7 of metalloporphyrins with an ammonia content of 200 ppb in the gas mixture.

As an example, FIG. 8 shows the result of applying the LDA method for gas mixtures containing one of the gases: NH₃, H₂S, NO₂, Et-SH. The points corresponding to different gases are grouped into regions on the LDA diagram, and the indicated regions are separated. In accordance with the specified algorithm in the microprocessor 12, for the obtained set Iᵣₑₗ^{k}, a point is calculated on the LDA diagram and the type of the detected connection is determined in relation to one of the regions.

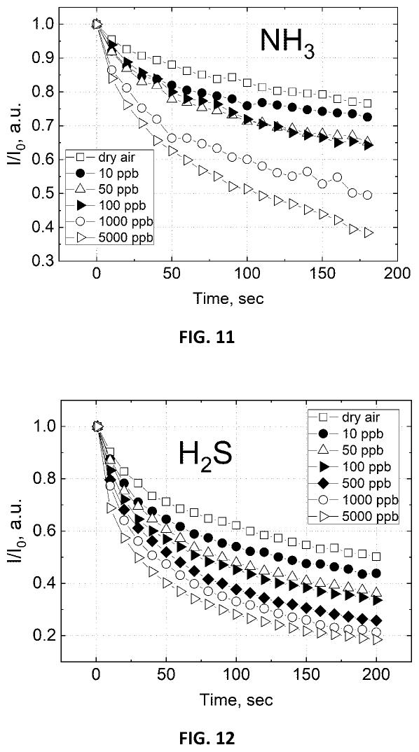

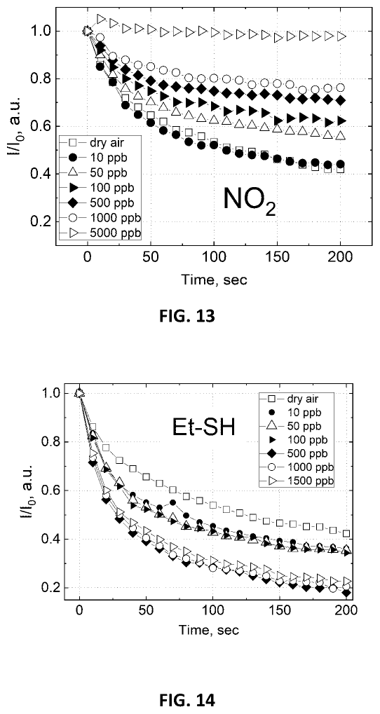

At the last step, according to the value Iᵣₑₗ^{k} obtained for the N organic field-effect transistor 1³ without a receptor layer, the concentration of the detected compound is determined according to calibration curves (FIGS. 11-14), previously stored in the memory of the microprocessor 12, according to the following algorithm. On the calibration curve of the current value versus concentration (FIG. 11-14), corresponding to the previously defined type of the detected compound, there is a point whose ordinate lies closest to the obtained value Iᵣₑₗ^{k}, and its abscissa is considered the measured concentration value. The established class of the detected compound, as well as its concentration measured in accordance with the algorithm, is then displayed to the user.

1. A gas multisensor, comprising an array of N organic field-effect transistors, each comprising at least “drain” and “source” electrodes separated by an organic semiconductor layer, a “gate” electrode, a dielectric layer and a receptor layer based on metalloporphyrin of general formula 1 or 2, completely or partially covering the organic semiconductor layer in a structure of the N-th organic field-effect transistor: 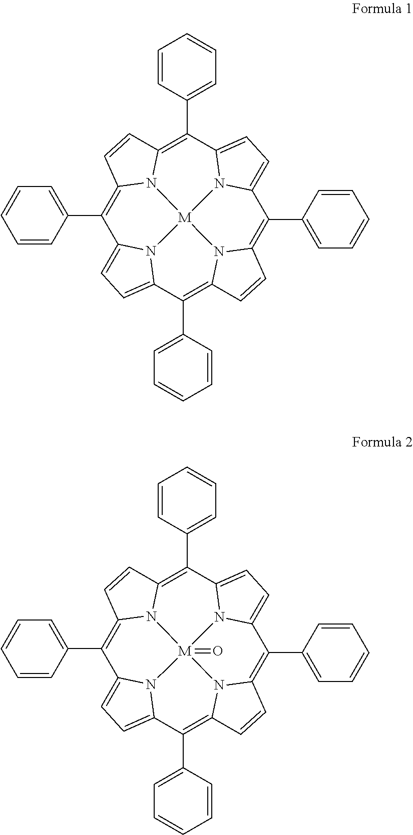
wherein the metal-ion M of metalloporphyrin is a transition metal, and each of the N organic field-effect transistors included in the array differs from other field effect transistors of the array by chemical structure of the receptor layer.

2. The gas multisensor according to claim 1, wherein the metal-ion M of metalloporphyrin is a transition metal ion selected from the following group: Cu, Zn, Co, Fe, Ni, Cr, Mn, Ti or V.

3. The gas multisensor according to claim 1, wherein the array of N organic field-effect transistors is formed on a single substrate.

4. The gas multisensor according to claim 1, wherein it determines the presence of one of the following gases: ammonia, hydrogen sulfide, nitrogen dioxide, ethyl mercaptan.

5. The gas multisensor according to claim 1, wherein the organic semiconductor layer of the organic field-effect transistor is a self-assembled monolayer made of chemically inert organosilicon derivatives of oligothiophene, benzothienobenzothiophene or diphenylbitiophene soluble in organic solvents.

6. The gas multisensor according to claim 1, wherein thickness of the organic semiconductor layer of the organic field effect transistor is 2-20 nm.

7. A gas multisensor, comprising an array of N organic field-effect transistors, each comprising at least “drain” and “source” electrodes, separated by an organic semiconductor layer, a gate electrode, a dielectric layer and a receptor layer based on metalloporphyrin of general formula 1 or 2, completely or partially covering the organic semiconductor layer in a structure of the N-th organic field-effect transistor: 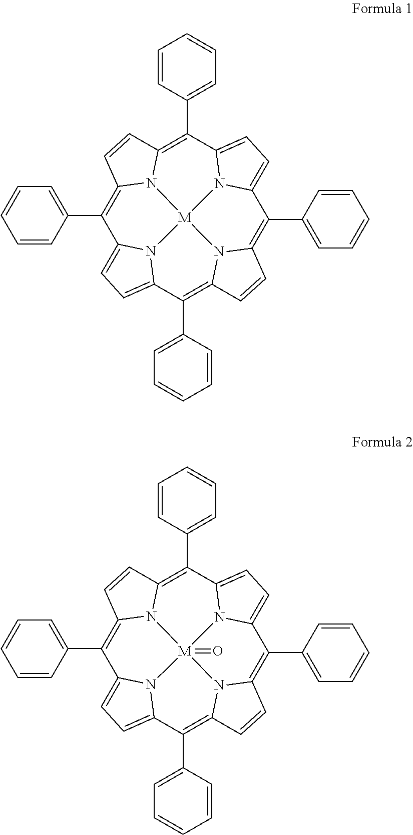
where the metal-ion M of metalloporphyrin is a transition metal, and each of the N organic field-effect transistors included in the array differs from other organic field-effect transistors in the array by chemical structure of the receptor layer, wherein the array of N organic field-effect transistors further comprises at least one organic field-effect transistor not having the receptor layer and suitable for quantitative determination of concentration of several low molecular weight toxic gases in atmospheric air or in a gas mixture.

8. The gas multisensor according to claim 7, wherein the metal-ion M of porphyrin is a transition metal ion selected from the following group: Cu, Zn, Co, Fe, Ni, Cr, Mn, Ti or V.

9. The gas multisensor according to claim 7, wherein after the array of N organic field-effect transistors is formed on a single substrate, organic field-effect transistors with the receptor layer are placed along perimeter of the substrate, and an organic field-effect transistor that does not have a receptor layer is located in central parts of the substrate.

10. The gas multisensor according to claim 7, wherein the receptor layers of metalloporphyrins are deposited using the Langmuir-Blodgett method by partially dipping the substrate into a subphase.

11. The gas multisensor according to claim 7, wherein the organic semiconductor layer of the organic field-effect transistor is a self-assembled monolayer made of chemically inert organosilicon derivatives of oligothiophene, benzothienobenzothiophene or diphenylbitiophene soluble in organic solvents.

12. The gas multisensor according to claim 7, wherein thickness of the organic semiconductor layer of the organic field effect transistor is 2-20 nm.

13. The gas multisensor according to claim 7, wherein it determines presence and concentration of one of the following gases: ammonia, hydrogen sulfide, nitrogen dioxide, ethyl mercaptan.

14. An “electronic nose” device for analyzing a multicomponent gas mixture, comprising:
a gas multisensor made according to claim 1 or claim 7;
a measuring unit connected to each of the organic field-effect transistors in the gas multisensor, the measuring unit being configured to measure current values in each of the N organic field-effect transistors included in the array depending on time;
a microprocessor connected to the measuring unit and configured to analyze response of each of the N organic field-effect transistors and determine a type of low molecular weight toxic gas present in the gas mixture, as well as its concentration;
a tight chamber with gas inlet and outlet, where the gas multisensor is located.

15. The device according to claim 14, wherein it determines presence and concentration of one of the following gases: ammonia, hydrogen sulfide, nitrogen dioxide, ethyl mercaptan.
